(19) United States
(12) Patent Application Publication
(10) Pub. No.: US 2008/0234669 A1
(43) Pub. Date: Sep. 25, 2008

(54) METHOD AND DEVICE FOR TREATING SKIN BY SUPERFICIAL COAGULATION

(76) Inventor: Arielle N.B. Kauvar, Scarsdale, NY (US)

Correspondence Address: MORGAN & FINNEGAN, L.L.P., 3 World Financial Center, New York, NY, 10281-2101, US

(21) Appl. No.: 11/687,710

(22) Filed: Mar. 19, 2007

(51) Int. Cl. A61B 18/20

(52) U.S. Cl. 606/9

(57) ABSTRACT

A method and system are disclosed for improving the appearance of skin utilizing an energy source, preferably a laser and more preferably an erbium laser device. The method includes using an energy source with or without pixilation adapted to be controlled by software or other means to achieve primarily coagulation to a limited depth of skin. Using this process, no appreciable ablation occurs and the superficial epidermal layer is not completely removed, thereby minimizing post-operative recovery time, patient pain, and post-operative risks.

### BACKGROUND OF THE INVENTION

Human skin damage or abnormalities can occur by many different means, for example, from aging, acne, trauma, scarring, photo-damage and other environmental injuries. Typically, these types of skin damage or abnormalities are associated with wrinkles (rhytides) and skin looseness, or laxity, which, biologically, result from changes in collagen, and elastin proteins and extracellular matrix (ECM). Collagen and elastin proteins are found in connective tissues that supply firmness and elasticity to the skin. When collagen, elastin and ECM within the dermal layer of the skin are degraded, weakened, or elongated, the skin becomes loose and wrinkles, lines, and depressions form.

There is a great interest among the general population and physicians in methods to ameliorate the damage to the skin. Chemical peeling and mechanical dermabrasion were the primary methods used to rejuvenate or resurface damaged skin prior to the 1990s. Although substantial improvement in skin appearance could be achieved with these methods, improvement was limited by the lack of depth control and unwanted side effects. Mechanical dermabrasion is a technique where the skin is literally sanded to a desired depth using abrasive materials and rotating skin abraders. The method is not well controlled and is difficult to use in some skin areas such as the eyelids. Overly deep treatment with either of these techniques produced untoward side effects including hypertrophic (thick) and atrophic (depressed) scarring, hypopigmentation (skin lightening), hyperpigmentation (skin darkening) as well as irregular textural changes. Because of these negative side effects and the difficulty with controlling the therapy, physicians investigated different methods for treating skin, including laser therapy.

High energy, short pulsed carbon dioxide (CO₂) lasers were the first lasers used for skin resurfacing, and have provided well established results (Waldorf H A, Kauvar A N, Geronemus R G. Skin resurfacing of fine to deep rhytides using a char-free carbon dioxide laser in 47 patients. Dermatol Surg. 1995 November; 21(11):940-6. Fitzpatrick, R E. CO₂ laser resurfacing. Dermatol Clin 2001; 19(3):453-456; Fitzpatrick, R E. Maximizing benefits and minimizing risk with CO₂ laser resurfacing. Dermatol Clin 2002; 20(1):77-86). Resurfacing CO₂ lasers use short pulse durations and sufficient energy, density or fluence to produce predictable depths of skin tissue removal (ablation) to a depth of ˜100-200 microns and underlying dermal thermal injury, (coagulation) of ˜80-150 microns in depth (Kauvar A N, Geronemus R G. Histology of laser resurfacing. Dermatol Clin. 1997 July; 15(3):459-67; Kauvar A N, Waldorf H, Geronemus R G. A histopathological comparison of “char-free” carbon dioxide lasers. Dermatol Surg. 1996 April; 22(4):343-8).

Removal or ablation of the damaged epidermis and variable depths of the dermis results in the formation of a new epidermis by means of wound healing and a new, thickened layer of collagen, producing an improved appearance of the skin. It is generally believed that coagulation of the papillary and superficial reticular dermis produced by the CO₂ laser is a primary cause for the observed improvement in wrinkles, scars and skin laxity (Ross, E V., et al. Why Does Carbon Dioxide Resurfacing Work? Arch Dermatol 1999; 135:444-454). Coagulation of a treated skin region results in collagen shrinkage and the formation of new collagen and elastin and, an immune response at the site of cellular damage. CO₂ laser treatment can produce long-term reduction in wrinkles and improvement in skin texture which are thought to be related to the formation of a new layer of collagen that replaces the damaged, elastotic tissue, as well as heat-induced shrinkage of the existing collagen fibrils (Ross, E V., et al. Why Does Carbon Dioxide Resurfacing Work? Arch Dermatol 1999; 135:444-454).

Although the clinical improvement in wrinkled, aged, and scarred skin by treatment with CO₂ lasers is often quite dramatic, such CO₂ laser treatments have been limited by the prolonged healing times and risk of adverse side effects. For example, CO₂ laser therapy requires patients to be anesthetized by general anesthetic or sedation for the treatment, which inherently creates additional risks for the procedure. Also, open wounds which result from this therapy often require up to 2 weeks to re-epithelialize and erythema (redness) of the skin can persist for months. Open wounds create risks for bacterial, viral, and fungal infections, and require after-treatment maintenance including cleaning and sterilization to prevent post-operative complications. Additionally, open wounds at the site of treatment, especially if the treatment is on the face, can create undesirable social and psychological affects. Patients are required to take time off from work and are reluctant to be seen in public until the wounds have healed. Other reported side effects related to treatment with CO₂ lasers include permanent skin hypopigmentation, hyperpigmentation, or scarring (Bernstein L J, Kauvar A N, Grossman M C, Geronemus. The short- and long-term side effects of carbon dioxide laser resurfacing. Dermatol Surg. 1997 July; 23(7):519-25; Nanni, C A and Alster, T S. Complications of Carbon Dioxide Laser Resurfacing. Dermatol Surg. An Evaluation of 500 Patients. 1998; 24(3):315-320). Therefore, while skin resurfacing can be achieved with CO₂ laser treatment, there are operative and post-operative risks and undesirable side effects.

The side effects and risks associated with CO₂ laser treatment led to further research with ablative laser systems. The Erbium:Yttrium-Aluminum-Garnet laser (Er:YAG; erbium) has been used as an alternative to the CO₂ laser. Initially, erbium lasers were used to create ablation of the skin with minimal or no coagulation. The erbium laser can be used to produce ablative wounds of 100-300 microns in depth which have been reported to heal faster and produce less erythema than comparable-depth ablation by CO₂ laser treatments by (Ross, R V, McKinlay J R, Sajben F P et al. Use of a novel erbium laser in a Yucatan minipig: a study of residual thermal damage, ablation, and wound healing as a function of pulse duration. Lasers Surg Med 2002; 30:93-100). While the erbium laser treatment reduces or obviates several of the side effects associated with CO₂ laser treatment, it has been reported that pure ablative erbium laser treatment often does not achieve the level of improvement to skin appearance that CO₂ laser treatment produces, nor does it result in the desired level of collagen shrinkage and increased collagen deposition (Kauvar A N B. Laser skin resurfacing: perspectives at the millennium. Dermatol Surg. 2000 February; 26(2):174-7; Ross, E V., et al. Why Does Carbon Dioxide Resurfacing Work? Arch Dermatol 1999; 135:444-454). Kauvar and Ross further reported that coagulation zones of treated skin at depths of at least 50-70 microns are required for efficient blood clotting to minimize undesirable bleeding and fluid draining at the site of treatment. Therefore, purely ablative erbium laser treatment produces less collagen production and collagen shrinkage, and ultimately less clinical improvement compared to treatments in which coagulation of the tissue occurs during treatment.

While purely ablative erbium laser treatments provide less than optimal skin resurfacing results, an advantage of erbium laser treatment is that it has a higher absorption coefficient for water and can be programmed to produce thin layers of ablation or coagulation by controlling various output parameters, including fluence, pulse-width, number of pulses, and frequency of pulses. By controlling these settings, the end user is capable of directing a specified amount of radiation to a limited area and depth of skin in order to optimize the range of ablation and coagulation. For example, dermal coagulation without complete epidermal ablation can be achieved by rapid stacking of low fluence erbium pulses. This effectively increases the pulse duration to achieve primarily coagulation. Heat transfer models show that extending the erbium laser pulse duration increases the depth of coagulation (Majaron, B., et al. Deep coagulation of dermal collagen with repetitive Er:YAG laser irradiation. Lasers Surg Med 2000; 26:214-221). Erbium lasers with longer pulse durations can produce coagulation depths similar to treatments utilizing CO₂ lasers (Ross, R V, McKinlay J R, Sajben F P et al. Use of a novel erbium laser in a Yucatan minipig: a study of residual thermal damage, ablation, and wound healing as a function of pulse duration. Lasers Surg Med 2002; 30:93-100), and comparable clinical efficacy is also seen (Rostan E F, Fitzpatrick R E, Goldman M P. Laser resurfacing with a long pulse erbium:YAG laser compared to a 950 ms pulsed CO₂ laser. Lasers Surg Med 2001; 29:136-41). However, when the erbium laser creates depths of both ablation and coagulation of the treated skin area similar to CO₂ laser treatment, it also produces similar undesired side effects (e.g. pain, edema, erythema, transudation, crusting and an increased risk of infection, scarring and pigment changes).

Other developments in laser skin treatment have involved the combining of erbium and CO₂ laser concepts to achieve the benefits from each laser type into one system. Three systems which have been reported include a hybrid, dual mode, and variable pulse system (Zachary, C B. Modulating the Er:YAG Laser. Lasers Surg Med. 2000; 26:223-226). The hybrid system is a combination of erbium and CO₂ lasers. The dual mode system comprises two erbium laser heads where one laser provides ablative pulses and the other laser provides coagulative pulses. The variable pulse system produces both ablative and coagulative pulses from one laser. While these modified erbium systems offer advantages over the separate traditional erbium and CO₂ lasers, patients are still left with prolonged open wounds, side effects similar to CO₂ laser treatment and may require several treatments to achieve the desired resurfacing results.

Non-ablative or noninvasive lasers or light sources have been reported as a better tolerated alternative to CO₂ and erbium laser treatments (Alam M, Tsu T S, Dover J S Wrone D A, Arndt K A. Nonablative laser and light treatments: histology and tissue effects—a review. Lasers Surg Med. 2003; 33(1):30-9). Such lasers and non-coherent light sources use visible and infrared wavelengths to heat the dermal layer to varying degrees without causing any appreciable damage to the epidermis. Many of these apparatuses use cooling methods to selectively cool the epidermis while heating the dermal tissue to induce new collagen production. These non-ablative devices, when used for the treatment of wrinkles, scars, and altered skin texture, require multiple treatment sessions and have little and unpredictable efficacy, compared to CO₂ and erbium lasers (Leffell D J. Clinical efficacy of devices for nonablative photorejuvenation. Arch Dermatol. 2002 November; 138(11):1503-8; Hardaway C A, Ross E V. Nonablative laser skin remodeling. Dermatol Clin. 2002 January; 20(1):97-111).

Recently, non-ablative lasers that can deeply penetrate dermal tissue have been used in a pixilated fashion to produce columns of coagulated tissue, typically 50-100 microns in diameter, with surrounding zones of unaltered tissue measuring 200-500 microns in diameter (Manstein D, Herron G S, Sink R K, Tanner H, Anderson R R. Fractional photothermolysis: A new concept for cutaneous remodeling using microscopic patterns of thermal injury. Lasers Surg Med. 2004; 34:426-38; Wanner M, Tanzi E L, Alster T S. Fractional photothermolysis: treatment of facial and nonfacial photodamage with a 1550 nm erbium-doped fiber laser. Dermatol Surg 007; 33:23-8). The zone of unaltered tissue between zones of treated tissue is termed the “pitch”. The pixilation technique exposes only a portion of the skin surface and volume to laser treatment, and the intervening zones of normal tissue prevent the development of full thickness or open wounds. Although better clinical results have been achieved with these devices compared to the non-ablative lasers, multiple treatment sessions utilizing this pixilated technique do not approach the superior results seen with a single deep erbium or CO₂ resurfacing treatment.

Another technique for skin rejuvenation uses the erbium laser in a purely ablative manner to peel away thin layers of the superficial portions of the epidermis, measuring 5-30 microns (Pozner J N, Goldberg D J. Superficial erbium:YAG laser resurfacing of photodamaged skin. J Cosmet Laser Ther. 2006 June; 8(2):89-91). These methods usually require multiple treatment sessions to achieve visible clinical improvement and while often reducing epidermal damage, do not improve wrinkles, scars, skin texture changes or skin laxity.

It is an object of the present invention to improve upon the shortcomings of the prior art and to describe a method for improving the appearance of skin by using superficial coagulation with or without superficial ablation of the damaged skin.

### BRIEF SUMMARY OF THE INVENTION

The present invention relates to a method for treating skin to improve its appearance comprising: identifying a region of a patient's skin having an undesirable or damaged condition suitable for treatment; exposing the skin tissue region to a controlled source of energy that heats the skin region and causes coagulation of a limited layer of the region of tissue located within the range of 0-250 microns from the skin surface, including the epidermis, without causing appreciable ablation of the epidermal skin in the region; allowing the patient's treated skin region to undergo healing; and observing an improvement in the appearance of the region of skin treated compared to the appearance of the skin region prior to treatment. Preferably the controlled source of energy is produce by a laser.

The present invention also relates to an erbium laser device having a control unit adapted to limit the output from the laser device to provide the parameters necessary to achieve coagulation of 0-250 microns of tissue from the skin surface without causing appreciable ablation and which can be used in the method described above.

The present invention also relates to other devices other than lasers, that can provide controlled noninvasive energy capable of providing to a limited target area of skin coagulation of 0-250 microns of tissue from the skin surface with no appreciable ablation.

### BRIEF DESCRIPTION OF THE DRAWINGS

FIG. 1. Histology from a skin tissue sample taken immediately after laser treatment of 2 pulses each set to deliver 25 microns of coagulation alone.

FIG. 2. Histology from a skin tissue sample taken immediately after laser treatment of 2 pulses each set to deliver 30 microns of ablation and 20 microns of coagulation.

FIG. 3. Histology from a skin tissue sample taken immediately after laser treatment of 2 pulses each set to deliver 30 microns of ablation alone.

FIG. 4. Histology from a skin tissue sample 24 hours after laser treatment of 3 pulses each set to deliver 20 microns of coagulation alone.

FIG. 5. Histology from a skin tissue sample 24 hours after laser treatment of 3 pulses each set to deliver 30 microns of ablation and 20 microns of coagulation.

### DETAILED DESCRIPTION OF THE INVENTION

The present invention provides a method for improving the appearance of damaged skin using an energy source. The invention also provides an improved laser device which is specifically adapted for controlling its output to achieve certain treatment results in the skin.

The method and improved laser of the present invention treats damaged skin having undesirable characteristics, which include, for example: a) discoloration due to brown spots such as freckles, seborrheic keratoses, patchy brown pigmentation, hyperpigmentation or melasma, sallow complexion or yellowing of the skin, dullness of the skin; b) enlarged capillaries or telengiectasia and skin redness; c) skin texture changes including enlarged pores, crepe appearance of thinned skin, skin growths such as enlarged oil glands (sebaceous hyperplasia) and other adnexal skin growths (e.g. syringoma, trichoepithelioma, angiofibroma); d) acne; e) fine and coarse wrinkling; f) skin laxity or looseness; g) scars from trauma, surgery or acne, including elevated and depressed scars. However, the invention is not limited to only treating these undesirable skin characteristics.

The invention provides a method which improves the appearance of skin by treating the skin tissue with an energy source to cause sufficient thermal injury to the epidermal and/or dermal layers of the skin. The process of causing thermal injury to cells or tissue without tissue vaporization or removal is referred to herein as coagulation. Tissue is coagulated when it reaches temperatures of approximately 70 degrees Celsius. At temperatures of approximately 70 degrees Celsius, collagen within the dermal layer is irreversibly damaged. Beneath this layer of coagulated collagen, there is a zone of collagen which is heated to approximately 55-60 degrees Celsius, in which type I collagen fibrils shrink to a third their length. The depth of dermal thermal damage is determined histologically by measuring the zone of basophilic change on sections stained with hematoxylin and eosin. In clinical laser treatments, coagulation of skin tissue typically causes shrinkage of existing collagen, the stimulation of new collagen production, and the induction of an immune response at and/or around the site of treatment (Ross, E V., et al. Why Does Carbon Dioxide Resurfacing Work? Arch Dermatol 1999; 135:444-454).

The stimulation of collagen can be observed on a microscopic level by analyzing a biopsy of the treated skin. Histology, immunohistochemistry, and electron microscopy of the sections of the skin tissue can assess the presence and level of collagen shrinkage, production and remodeling. On a macroscopic level, collagen shrinkage and stimulation can be observed as a reduction in fine and coarse wrinkles as well as increased tightening of the treated skin following laser therapy over time. Occasionally, the desired macroscopic results can be seen immediately following treatment of the affected area, however, the desired results may require several days, weeks, or even months to be noticeable. Since skin physiology varies from patient to patient, the time to achieve desired results may also vary.

The method and laser device of the present invention limit ablation of the skin region being treated to what is referred to herein as non-appreciable ablation. In contrast to coagulation, ablation typically results in destruction, removal, vaporization, or similar elimination of cells on the surface of the skin. Ablation is generally considered to be appreciable when it is associated with one or more of the following: complete removal of the epidermis with or without portions of the dermis; significant pain; and open wounds that are associated with fluid drainage, bleeding, crusting, or scabbing. Typically, following a treatment in which appreciable ablation of the skin occurs, extensive post-operative patient care is required in order to keep the wound occluded and moist to prevent infection and to assure proper healing. Open wounds can create a substantial risk for the patient to develop bacterial, viral, and fungal infections. It is necessary to occlude the open wounds with occlusive bandages or petroleum-based ointments and to keep the treated area clean and sterile in order to prevent infection.

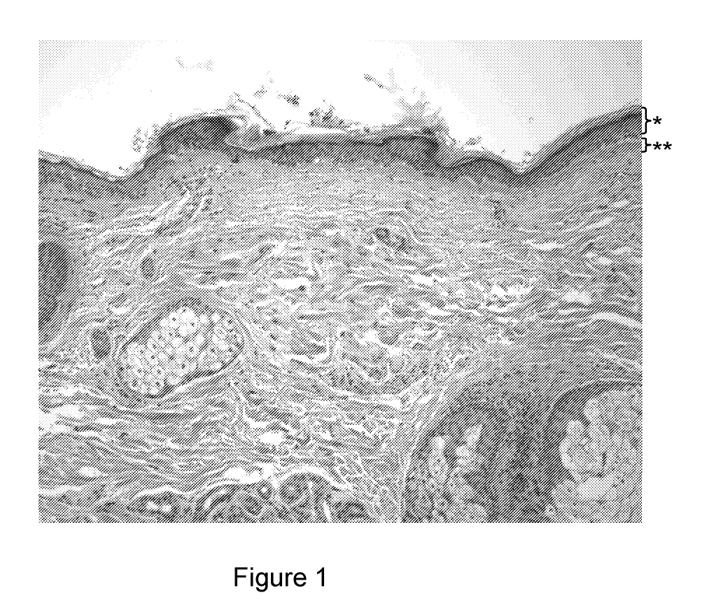

Non-appreciable ablation, on the other hand, does not result in significant pain and/or open wounds. Non-appreciable ablation can usually be achieved by limiting ablation of tissue to a skin layer depth of 50 microns or less from the skin surface. When purely coagulative pulses are applied to the skin, the epidermis develops partial or full thickness necrosis, but at least part of the epidermis remains intact, both clinically and in histological sections, in contradistinction to ablative pulses that produce immediate tissue removal without appreciable thermal alteration of the epidermis. See FIGS. 1-3.

In an embodiment of the invention, the energy source delivers adequate coagulation to the site of treatment, but does not cause appreciable ablation. In a preferred embodiment, an energy source such as a laser delivers coagulation from the surface of the epidermis to a depth of 130-250 microns with no appreciable epidermal ablation. In another preferred embodiment an energy source such as a laser delivers coagulation from the surface of the epidermis to a depth of 130-200 microns with no appreciable epidermal ablation

In another embodiment of the invention, the method is carried out without creation of an open wound at the site of treatment. The epidermal layer, which may exhibit partial necrosis, remains partially or wholly intact and acts as a natural bandage or barrier for the treatment area. This remaining superficial epidermal layer partially occludes the dermis, thereby minimizing fluid loss from the skin at the site of treatment. The partial occlusion protects the thermally damaged tissue from infection and minimizes patient pain, recovery time, and post-operative risks associated with the treatment. In an embodiment of the invention it is desirable for the method of treatment to cause some ablation of the epidermis but less than an appreciable ablation condition.

In an embodiment of the method of the present invention a step is included to contact the skin to be treated with a topical anesthetic prior to exposure to the energy source. The topical anesthetic is applied to the skin after cleansing with soap and water. The penetration of the topical anesthetic, and hence its effectiveness, can be improved by exfoliating the skin (i.e. removing the stratum corneum, or dead cell layer) by means of abrading it with a mechanical abrasion device, referred to as microdermabrasion, abrasive beads or application of an acid, such as glycolic or salicylic acid. Following the skin cleansing and/or exfoliation, the skin area is exposed to a topical anesthetic for varying times. The typical exposure time with the topical anesthetic is approximately 30-60 minutes. Typically, prior to treatment by the present method, the skin region which has been contacted with the topical anesthetic is removed with wet gauze and the skin is cleansed with isopropyl alcohol.

The method of the present invention includes a period of time for the treated skin region to undergo healing sufficient to observe improvement in the appearance of the treated skin region. The healing allows the biological effects of collagen shrinkage, new collagen generation, and/or the wound healing process to occur. During this period of time, microscopic and macroscopic improvements to the skin tissue structure and appearance can be observed. While improvement in the appearance of treated skin may be observed sooner, the period of time for substantially complete healing of the treated skin region can vary and range from up to one month, up to three months, and up to six months, depending on several factors, such as skin type, severity of skin damage, and age of the patient being treated, to name a few.

Furthermore, in another embodiment of the invention a step can be included to apply a topical substance to the skin region prior to, during or following treatment. The topical substance can provide one or more of several benefits including accelerating the healing process, reducing the severity of inflammation, discoloration and other side effects and boosting the wound healing response. The topical substance consists of, either singly or in combination, chemicals, compounds, growth factors, cytokines, and/or immune response modulators. The topical substance can include, but is not limited to: transforming growth factor-alpha, transforming growth factor-beta, granulocyte macrophage colony stimulating factor, connective tissue growth factor, epidermal growth factor, keratinocyte growth factor, platelet-derived growth factor, interleukin-1, interleukin-8, interleukin-28, interleukin-29, flavenoids, green tea extracts, obovatol, vitamin C, vitamin E, retinoids, calcitonin, parathyroid hormone-related protein, streptolysin O, nitric oxide, marinobufagenin, green tea extract; wound healing creams containing liquid paraffin, glycol monostearate, stearic acid, propylene glycol, paraffin wax, squalene, avocado oil, sweet almond oil, trolamine/sodium alginate, alginates, triethanolamine, cetyl palmitate, methylparaben (sodium salt), sorbic acid (as potassium salt), propyl paraben (sodium salt), and extracts from herbs, nuts, fruits and vegetables; cross-linked biopolymer gels and nanocellulose wound beds. The topical substance contributes to the treatment and healing process by stimulating and/or activating cells of the immune system including, but limited to, neutrophils, macrophages, myofibroblasts and/or fibroblasts at or around the treated skin region, in addition to stimulating the production of collagen, elastin and/or ECM.

In the method of the present invention, the treatment with an energy source can include one or more exposures or passes, such as multiple passes of a laser output over the treatment skin area. A laser pass typically refers to applying contiguous, minimally (20-50%) overlapping scans to an entire skin region, for example an entire upper lip or moustache area. Typically, passes in excess of one are determined based on the severity of the skin damage. Generally, treatment of a mild skin condition requires only one pass, while a more severe skin condition may require treatment of more than one pass. In preferred embodiments, the operator conducting the method of treatment subjects the skin treatment region to one to three passes of the energy source output.

In a preferred embodiment, the energy source of the invention is a laser. In a particular embodiment, the laser is any laser that has an absorption coefficient for water that is greater than or equal to 800 cm⁻¹. In another particular embodiment, the laser is a CO₂ or erbium laser device.

The laser output can be optionally delivered to the patient with a scanning handpiece. The scanning handpiece is held perpendicular to the skin area to be treated. The scanning handpiece is held so that the laser beam is focused on the skin being treated, or a within a distance in which the laser beam remains collimated. Alternatively, the laser output can be delivered through the lens of the laser which delivers radiation to a spot size of 0.5-20 mm, and/or a scanned pattern comprised of individual fixed diameter beams, typically 3-6 mm, and/or a pattern with a typical diameter of 3-20 mm created by an opticomehenical flashscanner, herein referred to as a flashscanned beam. In an embodiment, the flashscanned laser beam can be created by focusing a laser beam to a small spot size, typically 100-200 microns, which is scanned in a predetermined pattern, at a fixed rate, to create an area of tissue exposed to contiguous linear, curvilinear or spiral paths of the focused beam, such that any given point of tissue is exposed for a predetermined length of time to the laser output, so that the dwell time or exposure time of the laser beam is equivalent to the desired pulse width parameters for the laser exposure.

The laser device can be controlled to deliver to the skin region predetermined values for one or any combination of pulse number, pulse duration, frequency, spot size, fluence and exposure time to provide coagulation without causing appreciable ablation. In an embodiment, the pulse duration is within the range of 100-700 microseconds. In another embodiment, the pulse number is within the range of 2-20 pulses. The pulses can be delivered as a macropulse or in a train as a series of pulses. In another embodiment, the frequency is within the range of 10-50 Hz. In another embodiment, the fluence is within the range of 0.5-2.5 J/cm². In another embodiment, the spot size or scanned spot is within the range of 2-20 mm. In a further embodiment, the exposure time is within the range of 1-100 milliseconds.

In an embodiment, a laser device is adapted to be controlled by software or other means, which include, but are not limited to, digital/analog controls, a user interface control panel, a touch screen, and the like, which allow the operator to either adjust or select a fixed pre-set programmed control to the apparatus. The user interface control panel consists of a touch screen which allows the user to input/program settings for the laser output and a display which provides information to the user, such as pulse, frequency, fluence, and depth of ablation and/or coagulation. The software or other control means correlates treatment effect in terms of desired depths of coagulation and ablation to various output parameters of the laser or light-energy device, such as fluence, pulse frequency, pulse duration, and the like.

In another embodiment of the invention, the method and/or laser can be used with or without pixilation. Pixilation generally refers to the targeting of only a fraction of a treatment area at a time. This process can be achieved using a microlens array, hologram lens, a scanned laser beam, movement of the laser device by the operator, and the like. Pixilation typically works by the following concept: for any given beam or scan size, only a fraction of that beam or spot size is delivered to the tissue. In this way, microscopically or macroscopically small volumes of tissue of a determined diameter are exposed to the laser or energy source, with a constant distance of unaffected tissue between individual micro-beams. For each zone the laser or energy source targets and treats, the surrounding tissue is left unaffected and intact. In this way, pixilation allows treated skin to heal much faster than if the entire surface or volumetric area were treated at once. In an embodiment, the pixilation produces micro-beams of 50-2000 microns with a pitch of 50-2000 microns.

Pixilation can be achieved for the method using an energy source or laser that delivers energy to a region of skin that is simultaneously being cooled in a pixilated or fractionated fashion. In this way, only a fraction of the skin surface and volume being treated will be heated by the energy source. In a preferred embodiment, the cooling is delivered at equidistant intervals to the skin surface, with a typical pitch of 300-2000 microns. The pixilated cooling can be achieved, for example, by cooling a chamber at the tip of the laser handpiece that comes in contact with the skin, typically sapphire, chilled with water or cryogen spray.

The present invention includes a specially pre-set erbium laser device and a method for using such device. In an embodiment of the present invention an erbium laser device is controlled to deliver sufficient coagulation without causing appreciable ablation. The erbium laser device can be activated in a pulsed or scanned fashion, and can be controlled to deliver predetermined values for pulse duration, frequency, spot size, fluence, and the like, wherein the laser supplies an output such that the laser radiation is absorbed by the tissue and produces coagulation of tissue, including the epidermis and superficial dermis, limited to a depth of 250 microns or less and ablation of the epidermis limited to 50 microns or less. In an embodiment, the settings of the laser or energy source produces coagulation to a depth of 100-200 microns from the skin surface with no appreciable ablation of the epidermis.

The erbium laser of the present invention and the erbium laser as used in the method of the present invention can be a dual mode erbium laser capable of delivering radiation sufficient to cause ablation and/or coagulation. The variable pulsed erbium laser may comprise two erbium laser heads, in which one head delivers short-pulse and the other head delivers long-pulse components or one head that alternates between delivering ablative and coagulative pulses. The fluence, pulse frequency, pulse duration, and the like of the laser are capable of being controlled by the user through software or other means. In an embodiment, the dual-mode laser output parameters are controlled by a software which allows the user to adjust the laser device in a manner to achieve tissue coagulation from the epidermal surface in increments of 5 microns without appreciable ablation.

The invention is further explained or illustrated in the following non-limiting exemplary embodiments.

### EXAMPLE

### Assessment of Subjects to be Treated by the Claimed Method

Subjects with skin damage in the facial region were chosen to be treated with an erbium laser. The following types of skin conditions have been treated with this method: a) discoloration due to brown spots such as freckles, seborrheic keratoses, patchy brown pigmentation, hyperpigmentation or melasma; sallow complexion or yellowing of the skin; dullness of the skin; b) enlarged capillaries or telengiectasia and skin redness; c) skin texture changes including enlarged pores, crepe appearance of thinned skin, skin growths such as enlarged oil glands (sebaceous hyperplasia) and other adnexal skin growths (e.g. syringoma, trichoepithelioma, angiofibroma); d) fine and coarse wrinkling; e) skin laxity or looseness; f) scars form trauma, surgery or acne, including elevated and depressed scars.

### Description of the Erbium Laser System

To demonstrate the claimed method, subjects with damaged skin were treated with a variable pulse erbium laser (CONTOUR, by Sciton, Palo Alto, Calif.) equipped with dual erbium laser heads.

The laser consists of a modular platform, two 2940-nm Er:YAG lasers, dual pulse modes, a user interface control panel, a flat-top beam profile, a self-aligning articulated arm, a Large Area Pattern Generator (LAPG, by Sciton, Palo Alto, Calif.). The modular platform allows the user to use either one or dual erbium laser heads. The user modes, displayed on touch screens available from the manufacturer are: MicroLaserPeel, Laser Resurfacing and Single Spot. The Laser Resurfacing mode restricts the user to adjust the laser output to deliver a sufficient amount radiation to deliver tissue ablation from 0-100 microns with or without coagulation beneath the ablated tissue to a depth of 25, 50, 75 or 100 microns. The MicroLaserPeel mode restricts the user to adjust the laser output to deliver a sufficient amount radiation to cause skin ablation limited to depths of either 10, 20 or 30 microns. The laser can deliver a power up to 45 Watts. The laser delivers beams of radiation which have a pulse period ranging from 100 μs to 100 ms. The user interface control panel consists of a touch screen which allows the user to input/program settings for the laser output and a display which provides information to the user, such as pulse, frequency, fluence, and depth of ablation and/or coagulation. The Large Area Pattern Generator is a computer-guided high speed scanner which directs the path of the laser beam to precise spots.

The commercial model of the laser can be programmed using the Laser Resurfacing mode to deliver tissue ablation from 0-100 microns with or without coagulation beneath the ablated tissue to a depth of 25, 50, 75 or 100 microns. Ablation and coagulation depths are controlled by a user using a touch screen on the user interface control panel. The control panel displays corresponding fluence values for ablation levels (based on a calculation of 4 microns/J/cm² of ablation), however the control panel does not display the fluence used for any depth of coagulation chosen when 0 microns of ablation is chosen. The user interface control panel also does not display the pulse duration for any depth of ablation or coagulation.

When a combination of ablation and coagulation is chosen, the laser produces an initial ablative pulse followed by a series of sub-ablative pulses. However, the user interface control panel does not provide the pulse durations used to achieve the varying depths of coagulation or ablation. Coagulation with the laser is achieved by delivering a series of non-ablative pulses. For example, the approximate macropulse durations are 2, 8, 32 and 100 ms for desired coagulation depths (as displayed on the user interface control panel) of 25, 50, 100 and 200 microns, respectively, according to Ross (Ross, R V, McKinlay J R, Sajben F P et al. Use of a novel erbium laser in a Yucatan minipig: a study of residual thermal damage, ablation, and wound healing as a function of pulse duration. Lasers Surg Med 2002; 30:93-100). The laser uses a scanner with a 4 mm beam that treats adjacent 4 mm circular zones of tissue in a square pattern of various sizes that have a 0-50% overlap.

For the method of the present invention, a software was specifically designed which allows the user to customize the MicroLaserPeel mode, thus giving the user greater control to deliver non-ablative outputs in addition to ablative outputs. This software enables the user to utilize the dual head erbium laser in such a manner as to deliver 0-20 microns of tissue coagulation in increments of 5 microns with or without 0-30 microns of tissue ablation.

### Clinical Results of a Preferred Embodiment

Subjects undergoing erbium laser treatment with this device had the entire facial skin treated with the exception of the eyelid skin. The eyes were protected with external metal ocular shields. Prior to treatment, a skin exfoliating procedure was performed, which consisted of a microdermabrasion. After the exfoliating procedure, a topical anesthetic cream containing 4% lidocaine, was applied to the facial skin for approximately 45 min. Immediately prior to exposure to the laser, the skin region which was treated with the topical anesthetic was cleansed with water and isopropyl alcohol.

A scanning handpiece was held perpendicular to and approximately 1 cm above the skin surface, using parameters chosen from the MicroLaserPeel touch screen, as indicated in Table 3. Adjacent laser scans were placed contiguously over the entire facial skin, with the exception of the eyelids. Areas with deeper wrinkles or scars were treated with a second or third laser pass. When ablation alone was used, there was a typical high-pitched popping sound characteristic of an ablative erbium laser. When coagulation was added there was a change in the character of the sound of the pulses, with lower pitched audible pulse trains. Skin that was treated with coagulation alone developed a powdery white color change immediately after laser pulse impact, whereas skin treated with ablation alone or ablation and coagulation became reddened, and often developed pinpoint weeping of clear (serous) fluid. There was no immediate bruising, blistering, bleeding or crusting of the skin treated with any of the above-mentioned parameters. Additional subjects were treated with 25 microns of coagulation alone (using the Laser Resurfacing touch panel) mode. They developed the powdery white color change as well as focal (1-2 mm) spots of “char” when multiple laser passes were performed. Immediately following treatment, ice packs were applied for 15 to 20 minutes. Following the treatment, the patients were instructed to apply cold water compresses to their skin for 5-10 minutes followed by the application of a petrolatum-based ointment 4-5 times per day.

Subjects were treated with 1-3 passes of the laser, wherein a laser pass refers to applying contiguous, minimally (20-50%) overlapping scans to an entire skin region, for example an entire upper lip or moustache area. A second pass consists of performing a second set of contiguous scans over the same region, and a third pass consists of a third set of scans over the same skin region. Subjects were treated with 1-3 laser passes according to the laser procedures outlined in Table 1. In brief, MicroLaserPeel panel settings were as follows: 1) 30 microns of ablation alone, 2) 30 microns of ablation with 10 microns of coagulation, 3) 30 microns of ablation with 20 microns of coagulation, 4) 20 microns of coagulation alone; or 5) 25 microns of coagulation alone on the standard Laser Resurfacing touch panel. Four subjects were treated with each set of parameters.

TABLE 1
Laser procedure and associated side effects

| | Ablation | Coagulation | Pain Level | Erythema/Edema | Mild Desquamation | Transudate & |
|---|---|---|---|---|---|---|
| Procedure # | (um) | (um) | (out of 10) | (Days) | (Days) | Bronzing (out of 5) |
| 1 | 30 | 0 | 1–2 | 1–2 | 1–2 | 1 |
| 2 | 30 | 10 | 3–4 | 2–4 | 1–2 | 2 |
| 3 | 30 | 20 | 5–6 | 3–5 | 1–2 | 3 |
| 4 | 0 | 20 | 3–4 | 3–4 | 1–2 | 1 |
| 5 | 0 | 25 | 3–4 | 3–4 | 1–2 | 1 |

The treatment side effects were assessed for each procedure at the time of treatment, and then at 1 week, 1 month, and 3 months and are displayed in Table 1. The short- and long-term clinical results were also assessed for each procedure. Results from 1 and 3 month post-treatment are summarized in Table 2. Notable improvement to the treated skin region was observed at the one month follow up. Typically, there was additional improvement at the three month follow up.

TABLE 2
Laser procedure results observed at 1 and 3 months post-treatment.

| | Improved | Improved | Improved | | Improved | Reduced | Reduced | |
|---|---|---|---|---|---|---|---|---|
| | Brown | Brown | Sallow | Improved | Skin Texture/ | Mild | Coarse | Skin |
| Procedure # | Pigmentation | Spots | Appearance | Scars | Pores | Wrinkles | Wrinkles | Tightening |
| 1 | Yes | Yes | Yes | No | Mild | No | No | No |
| 2 | Yes | Yes | Yes | No | Mild | Mild | No | No |
| 3 | Yes | Yes | Yes | Yes | Yes | Yes | Yes | Yes |
| 4 | Yes | Yes | Yes | Yes | Yes | Yes | Yes | Yes |
| 5 | Yes | Yes | Yes | Yes | Yes | Yes | Yes | Yes |

### Histology

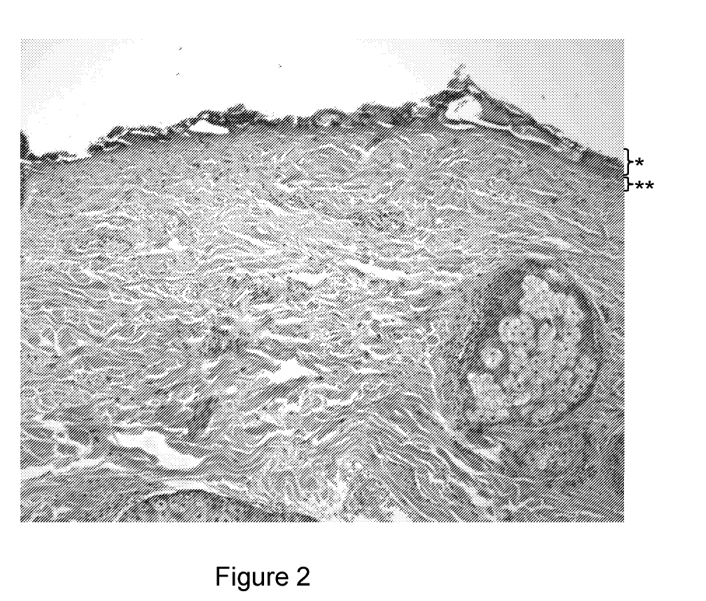

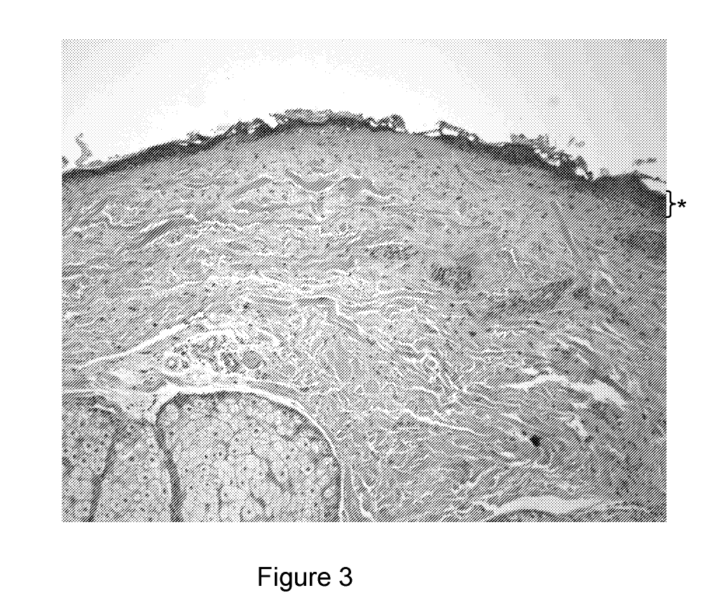

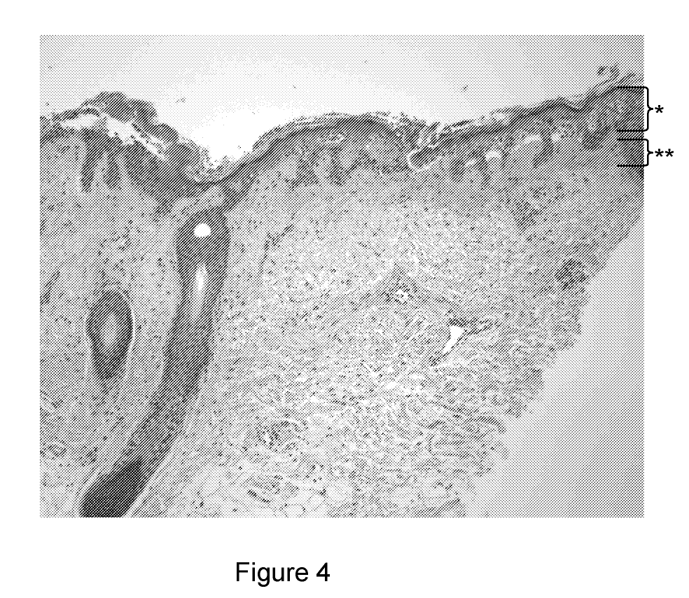

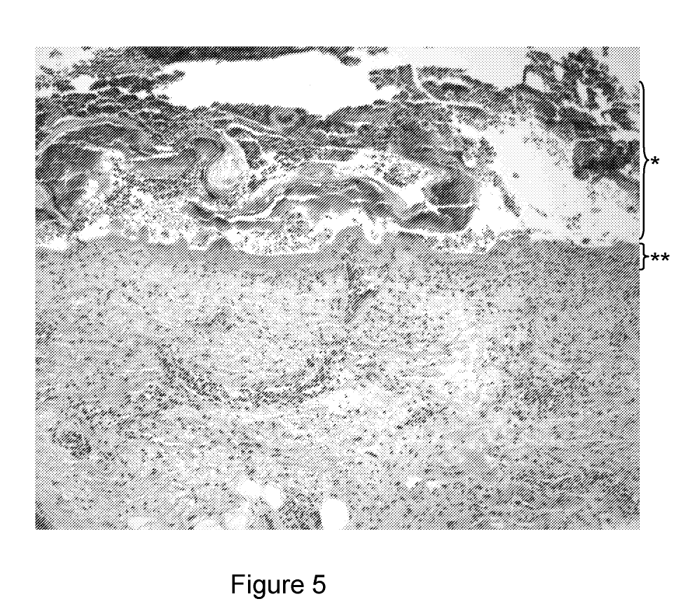

In addition to the macroscopic visual improvements to the skin surface, biopsies were taken from patients in order to assess the microscopic changes that occurred to the skin regions that were treated by the method. FIG. 1 shows that immediately after laser treatment with 2 pulses each set to deliver 25 microns of coagulation alone (Procedure 5) produced necrotic keratinocytes, but left an intact epidermis (*), and a 30-40 microns zone of dermal coagulation (**). FIG. 2 shows that immediately after laser treatment (Procedure 3) with 2 pulses each set to deliver 30 microns of ablation with 20 microns of coagulation produced vaporization or removal of approximately 40-100% of the epidermis (*), widespread necrosis in the residual epidermis, and a 20-30 microns zone of dermal coagulation (**). FIG. 3 shows that immediately after laser treatment (Procedure 1) with 2 pulses each set to deliver 30 microns of ablation alone produced vaporization or removal of approximately 40-100% of the epidermis (*), widespread necrosis in the residual epidermis, and no dermal coagulation. FIG. 4 shows the results 24 hours after laser treatment (Procedure 4) with 3 pulses each set to deliver 20 microns of coagulation alone. The epidermis is necrotic but present, there is a sparse mixed inflammatory infiltrate (*), and approximately 30 microns of dermal coagulation (**). FIG. 5 shows the results 24 hours after Procedure 3 with 3 pulses each set to deliver 30 microns of ablation and 20 microns of coagulation. The epidermis is necrotic and separated from the dermis (*), there is a dense mixed inflammatory infiltrate with neutrophils predominating, and approximately 30-40 microns of dermal coagulation (**).

### Patient Satisfaction

In addition to the biological evaluation of the skin following treatment, subjects were surveyed to determine their satisfaction of the procedure. Subjects treated with ablation alone (Procedure 1) were somewhat satisfied, subjects treated with ablation plus 10 microns of coagulation (Procedure 2) were moderately satisfied, and subjects treated with 20 microns of coagulation plus ablation (Procedure 3), 20 microns coagulation alone (Procedure 4), or 25 microns of coagulation alone (Procedure 5) were extremely satisfied. Three of these subjects (treated with procedure 4 or 5) had prior treatment with traditional CO₂ laser resurfacing and believed this procedure provided comparable results with a fraction of the downtime and recovery.

1. A method for treating skin to improve its appearance comprising:
identifying a region of a patient's skin having an undesirable or damaged condition suitable for treatment;
exposing the skin tissue region to a controlled source of energy that heats the skin region and causes coagulation of a limited layer of the region of tissue located within the range of 0-250 microns from the surface of the epidermis without causing appreciable ablation of the epidermal skin in the region;
allowing the patient's treated skin region to undergo healing; and
observing an improvement in the appearance of the region of skin treated compared to the appearance of the skin region prior to treatment.

2. The method of claim 1, wherein the identified region of the patient's skin for treatment possesses a condition selected from the group consisting of: discoloration, skin texture variations, enlarged pores, scarring, aging, photo-damage, wrinkles, trauma, acne and skin growths.

3. The method for treating skin according to claim 1, wherein the treated region develops no appreciable open wounds, draining, exudate, blistering or bleeding after exposure to the energy source.

4. The method of claim 1 wherein the portion of the skin tissue subject to coagulation is located in a layer within range of 100 to 200 microns below the treated skin surface.

5. The method of claim 1 wherein after exposure to the energy source an open wound is not created.

6. The method of claim 1 wherein the epidermal layer remains partially or wholly intact after exposure to the energy source.

7. The method of claim 1 wherein the region of skin treated compared to the appearance of the skin region prior to treatment comprises an improvement selected from the group consisting of: reduction in brown spots, reduction in discoloration, a reduction in acne, a reduction in wrinkles, a reduction in scars, a reduction in pore size, a reduction in skin dullness, a reduction in sallow complexion, a reduction in skin growths, and skin tightening.

8. The method of claim 1 wherein before or after exposure to the energy source, a topical composition is applied to skin region comprising one or more of cytokines, growth factors, and/or immune response modulators.

9. The method of claim 1 wherein the region of skin is exposed to repeated exposures of the energy source.

10. The method of claim 1 wherein the identified region of skin for treatment is contacted with a topical anesthetic prior to exposure to the energy source.

11. The method of claim 1 wherein the energy source is a laser.

12. The method of claim 11 wherein the energy source is a laser with a coefficient of absorption for water that is equal to or greater than 800 cm⁻¹.

13. The method of claim 11 wherein the energy source is a CO₂ or erbium laser device.

14. The method of claim 13 wherein the laser is an erbium laser device.

15. The method of claim 14 wherein the output of the erbium laser device is controlled to deliver to the skin region predetermined values for pulse number, pulse duration, frequency, spot size, and fluence to provide said coagulation without causing appreciable ablation.

16. The method of claim 14 wherein the erbium laser device delivers the energy through a scanning handpiece or through a handpiece with a fixed or variable spot size or flashscanned beam to the skin region.

17. The method of claim 12 wherein the laser output is delivered as a focused laser beam from a handpiece which is scanned over a predetermined area of tissue at a predetermined rate to achieve a specified tissue exposure time.

18. The method of claim 16 wherein the scanning or fixed or variable spot size or flashscanned beam handpiece delivers the energy in a series of equal laser pulses.

19. The method of claim 16 wherein the scanning or fixed or variable spot size or flashscanned beam handpiece delivers the energy in a series of varying laser pulses.

20. The method of claim 16, wherein the scanning or fixed or variable spot size or flashscanned beam handpiece is positioned perpendicular to the skin region.

21. The method of claim 16 wherein the fixed or variable spot size or flashscanned beam handpiece is position in the range of above the surface of the skin region in which the laser beam remains collimated.

22. The method of claim 15 in which the pulse duration is within the range of 100-700 microseconds.

23. The method of claim 15 in wherein the pulse number is within the range of 2-20 pulses.

24. The method of claim 15 wherein the frequency is within the range of 10-50 Hz.

25. The method of claim 15, wherein the fluence is within the range of 0.5-2.5 J/cm².

26. The method of claim 15, wherein the spot size or scanned spot is within the range of 2-20 mm.

27. The method of claim 15 wherein the pulse number is in the range of 2-20 pulses, the pulse duration is in the range of 100-700 microseconds and fluence of 0.5-2.5 J/cm² are delivered to each point of tissue with a frequency within a range of 10-50 Hz.

28. The method of claim 14 wherein the laser output is delivered as a focused laser beam from a handpiece which is scanned over a predetermined area of tissue at a predetermined rate to achieve a specified tissue exposure time of 1-100 milliseconds.

29. The method of claim 17 wherein the laser output has a fluence within the range of 0.5-2.5 J/cm².

30. The method of claim 17 wherein the exposure time is within a range of 1-100 msec and fluence is within the range of 0.5-2.5 J/cm².

31. The method of claim 27 wherein the output of the laser device is controlled by computer software programmed to control the output to predetermined values for pulse number, pulse duration, frequency, and fluence.

32. The method of claim 15, wherein the laser is a variable pulsed erbium laser with single or dual heads.

33. The method of claim 15, wherein the skin region is exposed to the output of the laser device in more than one pass.

34. The method of claim 15 wherein the exposure of the skin to the laser output is conducted by pixilating the laser beam.

35. The method of claim 15 wherein the exposure of the skin to the laser output is conducted with pixilated skin cooling.

36. An erbium laser device having a control unit adapted to limit the output from the laser device to provide the following parameter ranges:
pulse duration of 100-700 microseconds with
pulse trains or macropulses consisting of 2-20 pulses of 100-700 microseconds
delivered at a frequency of 10-50 Hz
with a fluence of 0.5-2.5 J/cm²
and a spot size or scan size of 2-20 mm.

37. The erbium laser device according to claim 36 wherein the control unit is adapted with controls to enable the operator when using the device to treat skin, to vary parameters within the said parameter ranges to achieve skin tissue coagulation in the skin depth of 0-250 microns, which can be varied in increments of 5-20 microns within the skin depth range without appreciable ablation.

38. The erbium laser device according to claim 36, wherein the laser is a variable pulsed erbium laser with single or dual heads.

39. The erbium laser device according to claim 36, whereby the control unit has a touch screen.

40. The erbium laser device according to claim 36, wherein the laser is adapted to deliver pixilation.

41. The erbium laser device according to claim 36 having a scanning handpiece.

42. The erbium laser device according to claim 36 having a flashscanned handpiece.

43. The erbium laser device according to claim 36 having a handpiece with a fixed or variable spot size.

44. The erbium laser device of claim 36 wherein the output is delivered as a focused laser beam from a handpiece which is scanned over a predetermined area of tissue measuring 2-20 mm at a predetermined rate to achieve a specified tissue exposure time of 1-100 milliseconds and a fluence of 0.5-2.5 J/cm².

45. The erbium laser device according to claim 36, wherein the laser is adapted to deliver energy in conjunction with pixilated skin cooling.
